(19) United States
(12) Patent Application Publication
(10) Pub. No.: US 2026/0217530 A1
(43) Pub. Date: Jul. 30, 2026

(54) PROCESS FOR PREPARATION OF SULFAMOYL CHLORIDE

(71) Applicant: Firmenich SA, Satigny (CH)

(72) Inventors: Herve Mosimann, Satigny (CH); Randall Harvey, Satigny (CH)

(73) Assignee: Firmenich SA, Satigny (CH)

(21) Appl. No.: 19/146,807

(22) PCT Filed: Jan. 8, 2024
(86) PCT No.: PCT/EP2024/050269

§ 371 Date: Jul. 9, 2025

(30) Foreign Application Priority Data
Jan. 12, 2023 (EP) 23151277.3

(51) Int. Cl. C01B 21/096; C07C 303/36; C07C 311/30; C07D 211/32; C07D 213/52; C07D 285/15; C07D 417/12

(52) U.S. Cl. CPC C01B 21/096; C07C 303/36; C07C 311/30; C07D 211/32; C07D 213/52; C07D 285/15; C07D 417/12

(57) ABSTRACT

The present invention relates to a process of preparing a compound having structural formula (I) comprising reacting a compound having structural formula (II) with water in the presence of an organic amide or an organic amine and to the use of a compound having structural formula (I) for the preparation of a compound having structural formula (IVa), (IVb) or (IVc).

### TECHNICAL FIELD OF THE INVENTION

The present invention relates to a process for preparing a compound having structural formula (I). More specifically, the present invention concerns a process for preparing a compound of formula (I) comprising reacting a compound of formula (II) with water in the presence of an organic amide or an organic amine. Furthermore, the present invention concerns the use of a compound having structural formula (I) for the preparation of a compound having the structural formula (IVa), (IVb) or (IVc).

### BACKGROUND OF THE INVENTION

Sulfamoyl chloride is not commercially available and is used for the synthesis of a variety of more complex compounds in different fields including, amongst others, nutrition, flavor, perfumery, cosmetics or pharmaceutics, and, in particular, taste modifiers, e.g. sweetening enhancers, sucrose enhancers and sweet flavor modifiers.

Sulfamoyl chloride is typically obtained from chlorosulfonyl isocyanate. Unfortunately, hydrolysis of chlorosulfonyl isocyanate is difficult to control and of low selectivity. The process most often found in the literature for the synthesis of sulfamoyl chloride uses the reaction of chlorosulfonyl isocyanate with formic acid in methylene chloride (CH₂Cl₂). This reaction is easier to control, but the reaction proceeds through two intermediates, an anhydride and a carbamic acid. The transformation of the anhydride to the carbamic acid involves the loss of one molecule of carbon monoxide and the conversion of the carbamic acid to sulfamoyl chloride releases one equivalent of carbon dioxide. Thus, two equivalents of gas are formed, one of which, namely carbon monoxide, is toxic. Other disadvantages of the known process are the use of chlorinated volatile solvents such as methylene chloride (CH₂Cl₂) and the use of formic acid, which is toxic, flammable, corrosive, unstable and decomposes to toxic carbon monoxide and water.

Consequently, there is a need for an improved process of preparing sulfamoyl chloride, i.e. a compound having structural formula (I), which is safe, environmentally friendly and scalable for industrial application.

The present invention solves the above problem by using an organic amide or an organic amine as a catalyst in order to prepare a compound of formula (I), i.e. sulfamoyl chloride, from reacting a compound of formula (II), i.e. chlorosulfonyl isocyanate, with water. To the best of our knowledge, the present invention's process has never been reported in the prior art.

### BRIEF DESCRIPTION OF THE FIGURES

FIG. 1: CO₂ release profile in the absence of dimethylacetamide. Conditions: 2-hour addition time of 1 mol eq. of water (10% w/w in acetonitrile) at 5° C. into a 50% w/w solution of chlorosulfonyl isocyanate in toluene. Post-addition temperature 5° C.

FIG. 2: CO₂ release profile in the presence of dimethylacetamide or triethylamine. Conditions: 2-hour addition time of 1 mol eq. of water (10% w/w in acetonitrile) containing 0.005 mol eq. of dimethylacetamide or triethylamine at 5° C. into a 33% w/w solution of chlorosulfonyl isocyanate in toluene. Post-addition temperature 25° C.

### DETAILED DESCRIPTION OF THE INVENTION

Surprisingly, it has now been discovered that reacting a compound of formula (II) with water in the presence of an organic amide or an organic amine allows the safe and scalable preparation of a compound of formula (I). The present invention's process is advantageously amenable to large scale processes.

A first object of the present invention is a process of preparing a compound having structural formula (I)

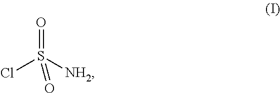

comprising reacting a compound having structural formula (II)

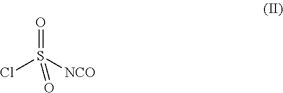

with water in the presence of an organic amide or an organic amine.

For the sake of clarity, by the expression “organic amide”, it is meant a compound having the general formula RC(═O)NR′R″, wherein R, R′, and R″, each independently, may represent hydrogen, alkyl, alkenyl, aryl, heteroaryl, heteroalkyl, arylalkyl, or heteroarylalkyl.

For the sake of clarity, the term “alkyl” as used herein has the general understanding by a skilled person. The term alkyl is herein particularly understood as linear, branched or cyclic C₁ to C₁₀ alkyl, particularly linear, branched or cyclic C₁ to C₈ alkyl, more particularly linear, branched or cyclic C₁ to C₄ alkyl, optionally substituted with a hydroxy group, C₁-C₃ alkoxy group or C₁-C₃ alkyl group such as for example methyl, ethyl, propyl, isopropyl, butyl or isobutyl.

For the sake of clarity, the term “alkenyl” as used herein has the general understanding by a skilled person. The term is herein alkenyl understood as linear, branched or cyclic C₂ to C₁₀ alkenyl, particularly linear, branched or cyclic C₂ to C₅ alkenyl, more particularly linear, branched or cyclic C₂ to C₄ alkenyl, each optionally substituted with a hydroxy group, C₁-C₃ alkoxy group or C₁-C₃ alkyl group, such as for example vinyl, allyl, propenyl, isopropenyl, butenyl or isobutenyl.

For the sake of clarity, the term “alkynyl” as used herein has the general understanding by a skilled person. The term is herein alkynyl understood as linear, branched or cyclic C₂ to C₁₀ alkynyl, particularly linear, branched or cyclic C₂ to C₈ alkynyl, more particularly linear, branched or cyclic C₂ to C₄ alkynyl, each optionally substituted with a hydroxy group, C₁-C₃ alkoxy group or C₁-C₃ alkyl group.

For the sake of clarity, the term “aryl” as used herein has the general understanding by a skilled person. The term aryl is herein particularly understood as Ce to C₁₀ aryl, optionally substituted with a hydroxy group, C₁-C₃ alkoxy group or C₁-C₃ alkyl group, such as for example phenyl.

For the sake of clarity, the term “heteroaryl” as used herein has the general understanding by a skilled person. The term heteroaryl is herein particularly understood as a C₄ to C₁₀ aryl group with at least one heteroatom such as nitrogen or oxygen in the ring moiety of the aryl group, optionally substituted with a hydroxy group, C₁-C₃ alkoxy group or C₁-C₃ alkyl group, such as for example pyridine or pyrimidine.

For the sake of clarity, the term “heteroalkyl” as used herein has the general understanding by a skilled person. The term heteroalkyl is herein particularly understood as a linear, branched or cyclic alkyl group with at least one heteroatom such as nitrogen, oxygen or sulfur in the alkyl moiety, in particular linear, branched or cyclic C₁ to C₁₀ heteralkyl, particularly linear, branched or cyclic C₁ to C₅ heteroalkyl, more particularly linear, branched or cyclic C₁ to C₄ heteroalkyl, optionally substituted with a hydroxy group, C₁-C₃ alkoxy group or C₁-C₃ alkyl group.

For the sake of clarity, the term “heteroalkenyl” as used herein has the general understanding by a skilled person. The term heteroalkenyl is herein particularly understood as a linear, branched or cyclic alkenyl group with at least one heteroatom such as nitrogen, oxygen or sulfur in the alkenyl moiety, in particular linear, branched or cyclic C to C₁₀ heteralkenyl, particularly linear, branched or cyclic C₁ to C₅ heteroalkenyl, more particularly linear, branched or cyclic C₁ to C₄ heteroalkenyl, optionally substituted with a hydroxy group, C₁-C₃ alkoxy group or C₁-C₃ alkyl group.

For the sake of clarity, the term “arylalkyl” as used herein has the general understanding by a skilled person. The term arylalkyl is herein particularly understood as an aryl substituted with an alkyl as described herein-above.

For the sake of clarity, the term “heteroarylalkyl” as used herein has the general understanding by a skilled person. The term heteroarylalkyl is herein particularly understood as an heteroalyl substituted with an alky as described herein-above.

The term “optionally” denotes that a group can or cannot comprise a certain functional group of substituent.

Examples of organic amides include, but are not limited to, acetamide, dimethylacetamide, benzamide, dimethylformamide, and combinations thereof.

In a preferred embodiment, the organic amide is dimethylacetamide.

The term “organic amine” herein means a compound having structural formula N(R)₃, wherein each R, each independently, may represent hydrogen, alkyl, alkenyl, aryl, heteroaryl, heteroalkyl, arylalkyl, or heteroarylalkyl, or alternatively, two of R, together with the nitrogen atom to which they are attached, form a cyclic heteroalkyl ring. Particularly, the organic amine is a tertiary amine; i.e. R may represent alkyl, alkenyl, aryl, heteroaryl, heteroalkyl, arylalkyl, or heteroarylalkyl, or alternatively, two of R, together with the nitrogen atom to which they are attached, form a cyclic heteroalkyl ring.

Examples of organic amines include, but are not limited to tripropylamine, tributylamine, triethylamine, diisoproylethylamine (DIEA), morpholine, methyl piperidine, ethyl piperidine, propyl piperidine and combinations thereof.

In a preferred embodiment, the organic amine is triethylamine.

In a particularly preferred embodiment, the organic amide is dimethylacetamide or the organic amine is triethylamine.

In a further preferred embodiment, the organic amide or the organic amine is not added to the reaction mixture after the formation of sulfamoyl chloride.

According to any embodiment of the present invention, the organic amine or the organic amide is present in a catalytic quantity.

The presence of a catalytic quantity of an organic amide or an organic amine results in a smooth reaction with a constant and controllable CO₂ evolution, while the release of toxic carbon monoxide is prevented. The release of carbon monoxide is prevented due to the reaction with water. Thus, the process according to the present invention is much safer from a gas-release standpoint.

The CO₂ release profile allows conclusions as to the smoothness of the reaction of the invention's process and can be measured by any means known to the skilled person. Advantageously, the CO₂ release profile can be measured as the CO₂ flowrate with a calibrated mass flowmeter.

These advantages render the process according to the present invention safe and environmentally friendlier as compared to the processes described in the prior art.

According to a specific embodiment, the catalytic quantity of the organic amide or the organic amine is not more than 0.3 mol/mol chlorosulfonyl isocyanate, preferably not more than 0.1 mol/mol chlorosulfonyl isocyanate, more preferably not more than 0.07 mol/mol chlorosulfonyl isocyanate, more preferably not more than 0.05 mol/mol chlorosulfonyl isocyanate.

According to a specific embodiment, the catalytic quantity of the organic amide or the organic amine is 0.001 to 0.3 mol/mol chlorosulfonyl isocyanate, preferably 0.002 to 0.1 mol/mol chlorosulfonyl isocyanate, more preferably 0.003 to 0.07 mol/mol chlorosulfonyl isocyanate.

According to a specific embodiment, the catalytic quantity of the organic amide or the organic amine is 0.05 mol/mol chlorosulfonyl isocyanate, 0.005 mol/mol chlorosulfonyl isocyanate or 0.003 mol/mol chlorosulfonyl isocyanate.

The catalytic quantities of the organic amide or the organic amine as specified herein advantageously result in a compound having structural formula (I), which shows an improved performance in any subsequent reaction including but not limited to the conversion of a compound having structural formula (III) to a compound having structural formula (IV). A further advantage of catalytic quantities of the organic amide or the organic amine according to the present invention is an improved process in terms of the CO₂ gas release profile. Surprisingly, the presence of a catalytic quantity of an organic amide or an organic amine results in a smooth reaction with regard to gas release and heat development.

The heat release is a further indicator of the smoothness of the invention's process and can be readily observed by the skilled person by temperature measurement.

Advantageously, the release of toxic carbon monoxide is prevented by the present invention's process.

In a particular embodiment chlorosulfonyl isocyanate is present in an inert solvent, preferably an inert aromatic solvent, inert alkane solvent or inert halogenated solvent.

The process according to the present invention does not require handling of chlorinated volatile solvents such as methylene chloride (CH₂Cl₂) and, therefore, there are no regulatory constraints due to the use of methylene chloride (CH₂Cl₂).

For sake of clarity, by the term “inert solvent” it is meant a solvent that is chemically not reactive with the dissolved compound.

Examples of inert solvents according to the present invention are dichloromethane, C₆₋₁₀ aromatic solvents such as xylene; toluene or chlorobenzene, or C₅₋₁₂ hydrocarbon solvents such as hexane, heptane, or cyclohexane, preferably toluene or chlorobenzene.

In a particularly preferred embodiment, chlorosulfonyl isocyanate is present in toluene.

According to a specific embodiment, the concentration of chlorosulfonyl isocyanate in the inert solvent, preferably toluene or chlorobenzene, is from 25% w/w to 50% w/w, preferably, from 30% w/w to 40% w/w. In a particularly preferred embodiment, the concentration of chlorosulfonyl isocyanate in the inert solvent, preferably toluene or chlorobenzene, is 33% w/w.

According to a specific embodiment, the amount of inert solvent, preferably toluene or chlorobenzene, relative to chlorosulfonyl isocyanate is 0.1 to 3 mol/mol chlorosulfonyl isocyanate, preferably 0.2 to 2.95 mol/mol chlorosulfonyl isocyanate, more preferably 0.5 to 2.5 mol/mol chlorosulfonyl isocyanate, most preferably 1.0 to 2.0 mol/mol chlorosulfonyl isocyanate.

In any embodiment, water is introduced as a dilute solution in an inert solvent, preferably an inert polar solvent, more preferably an inert polar water miscible solvent. In a preferred embodiment, water is added as solution in acetonitrile.

Advantageously, the present invention's process prevents the large accumulation of unstable intermediates releasing gas when transformed to sulfamoyl chloride.

Particularly, the water concentration in the non-reacting solvent, preferably acetonitrile, is from 0.5% w/w to 20% w/w, preferably from 1% w/w to 18% w/w, more preferably from 1.5% w/w to 15% w/w, more preferably from 2% w/w to 13% w/w, more preferably from 2.5% w/w to 12% w/w.

According to a particularly preferred embodiment, the water concentration in the non-reacting solvent, preferably acetonitrile, is from 3% w/w to 12% w/w, preferably from 4% w/w to 10% w/w, most preferably 10% w/w.

According to a specific embodiment, the amount of water relative to chlorosulfonyl isocyanate is 0.5 to 1.5 mol/mol chlorosulfonyl isocyanate, preferably 0.95 to 1.05 mol/mol chlorosulfonyl isocyanate, most preferably 1 mol/mol chlorosulfonyl isocyanate.

According to one embodiment, the reaction temperature is −5° C. to 35° C., preferably 5° C. to 25° C., more preferably 5° C. to 20° C., most preferably 5° C. to 15° C.

By the term “reaction temperature” is meant the temperature during the addition of water in the non-reacting solvent, preferably acetonitrile, in the presence of the organic amide or organic amine to chlorosulfonlyl isocyanate in the inert solvent, preferably toluene or chlorobenzene.

In one embodiment, the post-addition temperature is 15° C. to 65° C., preferably 20° C. to 60° C. In a particular embodiment, the post-addition temperature is 25° C. to 60° C.

The term “post-addition temperature” as used herein denotes the temperature applied after 1 hour after the addition time of the water in acetonitrile. The reaction mixture is left at the reaction temperature during the addition time of water in acetonitrile and 1 hour after the addition time of water. The reaction mixture is then brought to the post-addition temperature. An increase of the post-addition temperature to 25° C. to 60° C. advantageously suppresses any gas release upon storage of the resulting compound having structural formula (I) and unexpectedly increases the performance of the compound having structural formula (I) in subsequent reactions including but not limited to the preparation of compounds having structural formula (IV) from compounds having structural formula (III).

In a preferred embodiment, the addition time of water in the non-reacting solvent, preferably acetonitrile, in the presence of the organic amide or organic amine to chlorosulfonlyl isocyanate in inert solvent, preferably toluene or chlorobenzene, is 1 hour to 8 hours, preferably 1 hour to 6 hours, more preferably 1 hour to 4 hours, most preferably 2 hours to 4 hours.

The term “addition time” as used herein is to be understood as the time in which water in the non-reacting solvent, preferably acetonitrile, is added to the reaction mixture.

The present invention's process for the preparation of a compound of formula (I) may be carried out under batch and/or continuous conditions. In a preferred embodiment, the process is carried out in a continuous manner.

According to a particular embodiment, the process is used for converting a compound having structural formula (III)

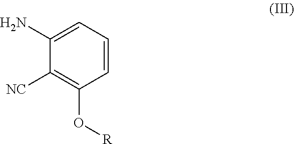

to a compound having a having structural formula (IV)

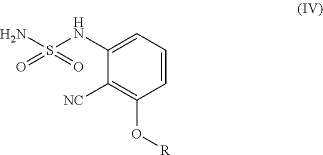

wherein R represents a C₁ to C₂₀ hydrocarbon group optionally comprising one or more heteroatoms such as oxygen, nitrogen and sulfur.

For the sake of clarity, the term “hydrocarbon” as used herein has the general understanding by a skilled person. The term hydrocarbon is understood in that said group consists of hydrogen and carbon atoms and can be in the form of a linear, branched or cyclic, aromatic, alkyl, alkenyl, or alkynyl group, e.g., a linear alkyl group, or can also be in the form of a mixture of said type of groups, e.g. a specific group may comprise a linear alkyl, a branched alkenyl (e.g. having one or more carbon-carbon double bonds), a (poly) cyclic alkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies.

For the sake of clarity, by the expression “optionally comprising one or more heteroatoms such as oxygen, nitrogen and sulfur”, or the similar, in the present invention it is meant that the group, to which is made reference, may comprise functional groups such as for examples amines, ethers, thioethers, acetals, esters, aldehydes, ketones, amides, carboxylates or alcohols.

In a particular embodiment, R represents C₁ to C₂₀ alkyl, alkenyl, alkynyl, heteroalkyl, heteroalkenyl or heteroaryl group or a C₁ to C₂₀ alkyl, alkenyl, alkynyl, heteroalkyl, heteroalkenyl or heteroaryl amid or ester group, optionally substituted with C₁ to C₂₀ alkyl, alkenyl, alkynyl, heteroalkyl, heteroalkenyl or heteroaryl group.

In a particular embodiment, the compound having the structure (III) is

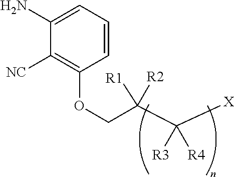

wherein n is 0 or 1;
R1 and R2 are, independently from each other, hydrogen atom or C₁ to C₄ alkyl group; or alternatively, R₁ and R₂, together with the carbon atom to which they are attached, form a C₃ to C₇ cycloalkyl;
R3 and R4 are, independently from each other, hydrogen atom or C₁ to C₄ alkyl group; or alternatively, R3 and R4, together with the carbon atom to which they are attached, form a C₃ to C₇ cycloalkyl;
X is a NRC(O)—R5 group wherein R is hydrogen or C₁ to C₆ alkyl; or R2 or R4 and R are taken together and form a C₃ to C₇ cycloalkyl and R5 is a C₁₋₆ alkyl, alkenyl or an aryl or heteroaryl or substituted heteroaryl; or
X is a C(O)NR6 wherein R6 is a C₁₋₆ alkyl, alkenyl or an aryl or heteroaryl or substituted heteroaryl.

In a particular embodiment, the compound having the structure (IV) is

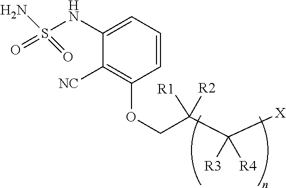

wherein n, R1, R2, R3, R4 and X are as described herein-above.

For the sake of clarity, particular embodiments for alkyl, alkenyl, alkynyl, heteroalkyl, heteroalkenyl or heteroaryl group or a corresponding a C₁ to C₂₀ alkyl, alkenyl, alkynyl, heteroalkyl, heteroalkenyl or heteroaryl amid or ester group are described herein above.

Thereby, the present invention also relates to a process for preparing a compound having structural formula (IV), the process comprises the steps of:
preparing a compound having structural formula (I) as disclosed herein-above,
reacting a compound having structural formula (I) with a compound having structural formula (III).

In a specific embodiment, the compound having structural formula (III) is

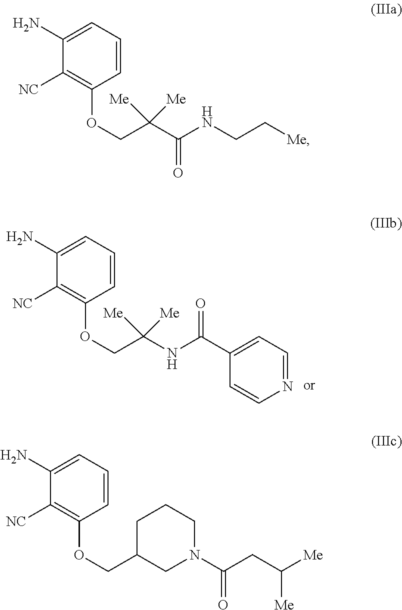 [SMILES: CCCNC(=O)C(C)(C)COc1cccc(N)c1C#N ; CC(C)(COc1cccc(N)c1C#N)NC(=O)c1ccncc1 ; CC(C)CC(=O)N1CCCC(COc2cccc(N)c2C#N)C1]

and the compound having structural formula (IV) is

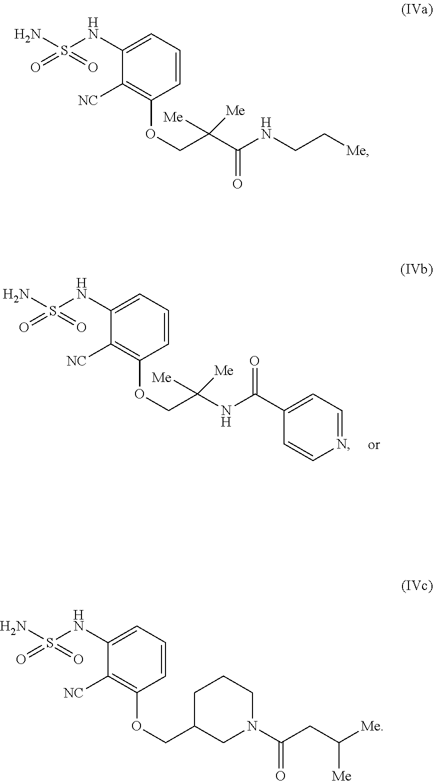 [SMILES: CCCNC(=O)C(C)(C)COc1cccc(NS(N)(=O)=O)c1C#N ; CC(C)(COc1cccc(NS(N)(=O)=O)c1C#N)NC(=O)c1ccncc1 ; CC(C)CC(=O)N1CCCC(COc2cccc(NS(N)(=O)=O)c2C#N)C1]

According to a particular embodiment, the compound of structural formula (IV) is converted, preferably in-situ or in a stepwise manner, to a compound of structural formula (V)

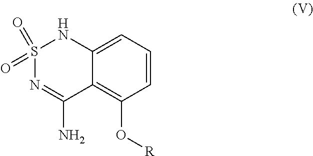

or any orally acceptable salt thereof and wherein R represents a C₁ to C₂₀ hydrocarbon group optionally comprising one or more heteroatoms such as oxygen, nitrogen and sulfur.

In a particular embodiment, R represents a C₁-C₂₀ alkyl, alkenyl, alkynyl, heteroalkyl, heteroalkenyl or heteroaryl group or a C₁-C₂₀ alkyl, alkenyl, alkynyl, heteroalkyl, heteroalkenyl or heteroaryl amid or ester group, optionally substituted with C₁-C₂₀ alkyl, alkenyl, alkynyl, heteroalkyl, heteroalkenyl or heteroaryl group.

In a particular embodiment, the compound having the structure (V) is

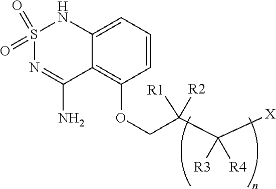

wherein n, R1, R2, R3, R4 and X are as described herein-above.

In an alternative embodiment, the compound of structural formula (IVa), (IVb) or (IVc) is converted, preferably in-situ or in a stepwise manner, to a compound of structural formula (Va), (Vb) or (Vc)

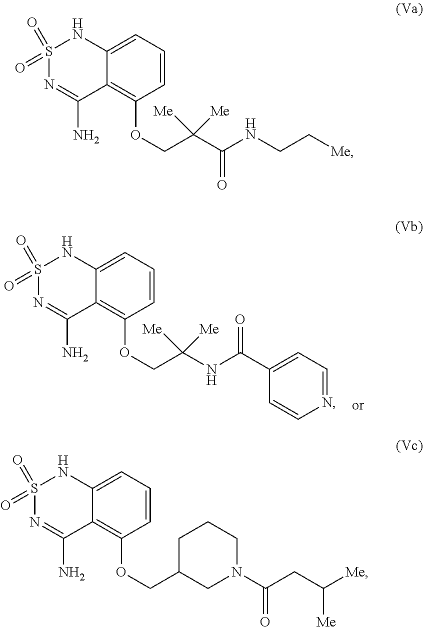 [SMILES: CCCNC(=O)C(C)(C)COc1cccc2c1C(N)=NS(=O)(=O)N2 ; CC(C)(COc1cccc2c1C(N)=NS(=O)(=O)N2)NC(=O)c1ccncc1 ; CC(C)CC(=O)N1CCCC(COc2cccc3c2C(N)=NS(=O)(=O)N3)C1]

or any orally acceptable salt thereof.

For the sake of clarity, the expression “in situ” is to be understood as “in the reaction mixture.”

In a particular embodiment, the compound having structural formula (V) is a taste modifier.

In a specific embodiment, the compound having structural formula (V) is a sweetener enhancer.

According to a preferred embodiment, the compound having structural formula (V) is a sucrose enhancer.

In a specific embodiment, the compound having structural formula (I) is used for converting a compound having structural formula (III) to a compound having structural formula (IV).

The yield of conversion from a compound having structural formula (III) to a compound having structural formula (IV) can be measured by any means known to the skilled person. Advantageously, the yield of conversion can be measured by high-liquid performance chromatography (HPLC).

In a further aspect, the present invention relates to the use of a compound having structural formula (I)

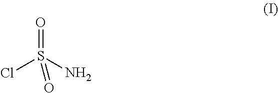

for the preparation of a compound having structural formula (IV) or (V), particularly to a compound having structural formula (IVa), (IVb) or (IVc)

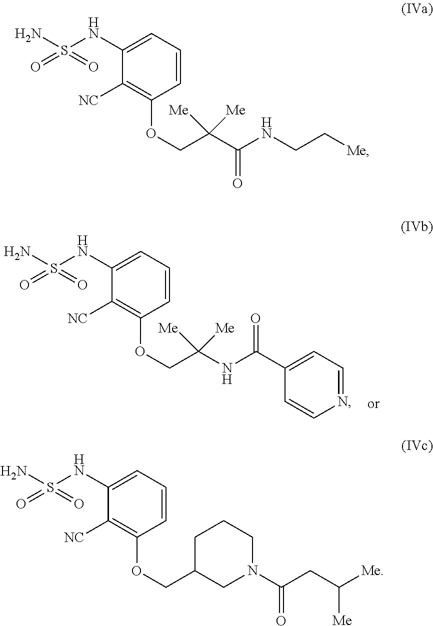 [SMILES: CCCNC(=O)C(C)(C)COc1cccc(NS(N)(=O)=O)c1C#N ; CC(C)(COc1cccc(NS(N)(=O)=O)c1C#N)NC(=O)c1ccncc1 ; CC(C)CC(=O)N1CCCC(COc2cccc(NS(N)(=O)=O)c2C#N)C1]

Typical manners to execute the invention's process are reported herein-below in the examples, which should not be considered as limiting the invention. In the examples, unless otherwise specified, the abbreviations have the usual meaning in the art, the temperatures are indicated in degrees Celsius (C).

### EXAMPLES

### Example 1

The equipment used was a 1-L automated reactor equipped with a condenser and a calibrated mass flowmeter for CO₂ flowrate measurement. The equipment was made of glass and Teflon.

Preparation of sulfamoyl chloride by reacting 1 mol equivalent (eq.) of water in acetonitrile (10% w/w in acetonitrile) and chlorosulfonyl isocyanate in toluene (50% w/w in toluene). The addition time of water in acetonitrile was 2 hours and the temperature during the addition of water was 5° C. Post-addition temperature was 5° C. The CO₂ release profile was used as indicator of the smoothness of the reaction.

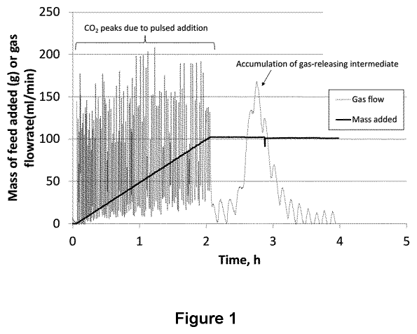

FIG. 1 shows the CO₂ release profile of example 1. The gas release profile consists of narrow peaks and a large CO₂ peak is suddenly generated about 15 minutes after the end of the addition. The sudden and uncontrolled gas release is not acceptable form a safety standpoint.

The thus prepared sulfamoyl chloride solution was used to convert a compound having structural formula (IIIa) to a compound having structural formula (IVa).

In a round bottom flask equipped with a magnetic stirrer, 2 g of a compound having structural formula (IIIa) (1 mol eq.), 0.34 g of dimethylacetamide and 4.48 g of acetonitrile were added. To this suspension, 1.4 mol eq. [mol sulfamoyl chloride/compound having structural formula (IIIa)] of the sulfamoyl chloride solution prepared as described above was added and left at 20° C. for 1 hour under stirring. To this reaction mixture 1.03 g (1.4 mol eq.) of triethylamine were added in 1 hour at 20° C. The yield of the compound having structural formula (IVa) in the crude sample was evaluated by high-performance liquid chromatography (HPLC).

The yield was 78.3%.

### Example 2

The equipment used was a 1-L automated reactor equipped with a condenser and a calibrated mass flowmeter for CO₂ flowrate measurement. The equipment was made of glass and Teflon.

Preparation of sulfamoyl chloride by reacting 1 mol equivalent (eq.) of water in acetonitrile (10% w/w in acetonitrile) and chlorosulfonyl isocyanate in toluene (33% w/w in toluene) in the presence triethylamine or dimethylacetamide (of 0.005 mol per mol of chlorosulfonyl isocyanate). The addition time of water in acetonitrile was 2 hours and the temperature during the addition of water was 5° C. Post-addition temperature was 25° C. The CO₂ release profile was used as indicator of the smoothness of the reaction.

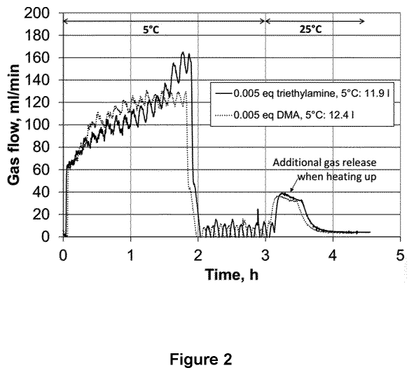

FIG. 2 shows the CO₂ release profile of example 2. With dimethylacetamide and triethylamine, the gas flowrate during the addition period is constant and smooth. There is no additional CO₂ release during the post-addition period at 5° C. Upon re-heating to 25° C. post-addition temperature, there is an additional amount of CO₂ released, which is significantly less than the accumulated gas released, if the process is performed in the absence of dimethylacetamide or triethylamine (FIG. 1), and, therefore safe for industrial application.

The thus prepared sulfamoyl chloride solution was used to convert a compound having structural formula (IIIa) to a compound having structural formula (IVa).

In a round bottom flask equipped with a magnetic stirrer, 2 g of a compound having structural formula (IIIa) (1 mol eq.), 0.34 g of dimethylacetamide and 4.48 g of acetonitrile were added. To this suspension, 1.4 mol eq. [mol sulfamoyl chloride/compound having structural formula (IIIa)] of sulfamoyl chloride solution prepared as described above was added and left at 20° C. for 1 hour under stirring. To this reaction mixture 1.03 g (1.4 mol eq.) of triethylamine were added in 1 hour at 20° C. The yield of the compound having structural formula (IVa) in the crude sample was evaluated by high-performance liquid chromatography (HPLC).

The yield was 73.4% using sulfamoyl chloride prepared in the presence of dimethylacetamide and 71.8% using sulfamoyl chloride prepared in the presence of triethylamine.

### Example 3

Sulfamoyl chloride was prepared in a 1500-liter enameled reactor. 1 mol equivalent (eq.) of water in acetonitrile (10% w/w in acetonitrile) and chlorosulfonyl isocyanate in toluene (33% w/w in toluene) were reacted in the presence of dimethylacetamide (0.003 mol per mol of chlorosulfonyl isocyanate). The addition time of water in acetonitrile was 4 hours and the temperature during the addition of water was 15° C. Post-addition temperature was 60° C.

To assess the regularity (smoothness) of the reaction, the heat release has been observed. The reaction showed a smooth heat release profile.

The sulfamoyl chloride solution was used to convert a compound having structural formula (IIIa) to a compound having structural formula (IVa). The yield of the compound having structural formula (IVa) in the crude sample was evaluated by high-performance liquid chromatography (HPLC) under the conditions as described in examples 1 and 2.

The yield of the compound having structural formula (IVa) was 69.2% with respect to the compound having structural formula (IIIa), indicating a robust process.

### Example 4

Sulfamoyl chloride was prepared in a 1500-liter enameled reactor. 1 mol equivalent (eq.) of water in acetonitrile (10% w/w in acetonitrile) and chlorosulfonyl isocyanate in toluene (33% w/w in toluene) were reacted in the presence of 0.003 mol/mol chlorosulfonlyl isocyanate dimethylacetamide (0.003 mol per mol of chlorosulfonlyl isocyanate). The addition time of water in acetonitrile was 4 hours and the temperature during the addition of water was 20° C. Post-addition temperature was 60° C.

To assess the regularity (smoothness) of the reaction, the heat release has been observed. The reaction showed a smooth heat release profile.

The sulfamoyl chloride solutions were used to convert a compound having structural formula (IIIa) to a compound having structural formula (IVa). The yield of the compound having structural formula (IVa) in the crude sample was evaluated by high-performance liquid chromatography (HPLC) under the conditions as described in examples 1 and 2.

The yield of the compound having structural formula (IVa) was 67.7% with respect to the compound having structural formula (IIIa), indicating a robust process.

### Example 5

Sulfamoyl chloride was prepared in a 1500-liter enameled reactor. 1 mol equivalent (eq.) of water in acetonitrile (10% w/w in acetonitrile) and chlorosulfonyl isocyanate in toluene (33% w/w in toluene) were reacted in the presence of dimethylacetamide (0.002 mol per mol of chlorosulfonlyl isocyanate). The addition time of water in acetonitrile was 4 hours and the temperature during the addition of water was 15° C. Post-addition temperature was 60° C.

To assess the regularity (smoothness) of the reaction, the heat release has been observed. The reaction showed a smooth heat release profile.

The sulfamoyl chloride solutions were used to convert a compound having structural formula (IIIa) to a compound having structural formula (IVa). The yield of the compound having structural formula (IVa) in the crude sample was evaluated by high-performance liquid chromatography (HPLC) under the conditions as described in examples 1 and 2.

The yield of the compound having structural formula (IVa) was 69.5% with respect to the compound having structural formula (IIIa), indicating a robust process.

### Example 6

Sulfamoyl chloride was prepared in a 1500-liter enameled reactor. 1 mol equivalent (eq.) of water in acetonitrile (10% w/w in acetonitrile) and chlorosulfonyl isocyanate in toluene (33% w/w in toluene) were reacted in the presence of dimethylacetamide (0.003 mol per mol of chlorosulfonly isocyanate). The addition time of water in acetonitrile was 5 hours and the temperature during the addition of water was 15° C. Post-addition temperature was 60° C.

To assess the regularity (smoothness) of the reaction, the heat release has been observed. The reaction showed a smooth heat release profile.

The sulfamoyl chloride solutions were used to convert a compound having structural formula (IIIa) to a compound having structural formula (IVa). The yield of the compound having structural formula (IVa) in the crude sample was evaluated by high-performance liquid chromatography (HPLC) under the conditions as described in examples 1 and 2.

The yield of the compound having structural formula (IVa) was 67.6% with respect to the compound having structural formula (IIIa), indicating a robust process.

### Example 7

The equipment used was a 1-L automated reactor equipped with a condenser and a calibrated mass flowmeter for CO₂ flowrate measurement. The equipment was made of glass and Teflon.

Preparation of sulfamoyl chloride by reacting 1 mol equivalent (eq.) of water in acetonitrile (10% w/w in acetonitrile) and chlorosulfonyl isocyanate in toluene (33% w/w in toluene) in the presence of dimethylacetamide (0.002 mol per mol of chlorosulfonyl isocyanate). The addition time of water in acetonitrile was 2 hours and the temperature during the addition of water was 5° C. Post-addition temperature was 60° C. The CO₂ release profile was used as indicator of the smoothness of the reaction.

The thus prepared sulfamoyl chloride solution was used to convert a compound having structural formula (IIIa) to a compound having structural formula (IVa).

The yield of the compound having structural formula (IVa) in the crude sample was evaluated by high-performance liquid chromatography (HPLC).

The yield was 78.2% using sulfamoyl chloride prepared in the presence of dimethylacetamide.

### Example 8

The equipment used was a 1-L automated reactor equipped with a condenser and a calibrated mass flowmeter for CO₂ flowrate measurement. The equipment was made of glass and Teflon.

Preparation of sulfamoyl chloride by reacting 1 mol equivalent (eq.) of water in acetonitrile (12% w/w in acetonitrile) and chlorosulfonyl isocyanate in toluene (33% w/w in toluene) in the presence of dimethylacetamide (0.001 mol per mol of chlorosulfonyl isocyanate). The addition time of water in acetonitrile was 1.5 hours and the temperature during the addition of water was 15° C. Post-addition temperature was 60° C. The CO₂ release profile was used as indicator of the smoothness of the reaction.

The thus prepared sulfamoyl chloride solution was used to convert a compound having structural formula (IIIa) to a compound having structural formula (IVa).

The yield of the compound having structural formula (IVa) in the crude sample was evaluated by high-performance liquid chromatography (HPLC).

The yield was 74.6% using sulfamoyl chloride prepared in the presence of dimethylacetamide.

1. A process of preparing a compound having structural formula (I) 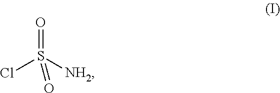
comprising reacting a compound having structural formula (II) 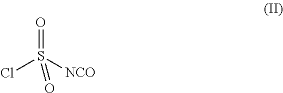
with water in the presence of an organic amide or an organic amine.

2. The process according to claim 1, wherein the organic amine or the organic amide is present in a catalytic quantity.

3. The process according to any of the preceding claims, wherein the organic amide is dimethylacetamide and the organic amine is triethylamine.

4. The process according to claim 3, wherein the catalytic quantity of the organic amide or the organic amine is 0.001 to 0.3 mol/mol chlorosulfonyl isocyanate, preferably 0.002 to 0.1 mol/mol chlorosulfonyl isocyanate, more preferably 0.003 to 0.07 mol/mol chlorosulfonyl isocyanate, more preferably 0.05 mol/mol chlorosulfonyl isocyanate, more preferably 0.005 mol/mol chlorosulfonyl isocyanate, most preferably 0.003 mol/mol chlorosulfonyl isocyanate.

5. The process according to any of the preceding claims, wherein chlorosulfonyl isocyanate is present in an inert solvent, preferably an inert aromatic solvent, inert alkane solvent or inert halogenated solvent.

6. The process according to claim 5, wherein chlorosulfonyl isocyanate is present in a C₆₋₁₀ aromatic solvent, dichloromethane, or a C₅₋₁₂ hydrocarbon solvent, wherein the C₅₋₁₂ hydrocarbon solvent is selected from the group of hexane, heptane, or cyclohexane, wherein the C₆₋₁₀ aromatic solvent is selected from the group of xylene; toluene or chlorobenzene, preferably, wherein the C₆₋₁₀ aromatic solvent is toluene or chlorobenzene, most preferably, wherein the C₆₋₁₀ aromatic solvent is toluene.

7. The process according to claim 6, wherein the concentration of chlorosulfonyl isocyanate in the inert solvent, preferably toluene or chlorobenzene, is from 25% w/w to 50% w/w, preferably 33% w/w.

8. The process according to any of the preceding claims, wherein water is added as solution in an inert solvent, preferably an inert polar solvent, preferably in acetonitrile.

9. The process according to claim 8, wherein the water concentration in acetonitrile is from 0.5% w/w to 20% w/w, preferably from 1% w/w to 18% w/w, more preferably from 1.5% w/w to 15% w/w, more preferably from 2% w/w to 13% w/w, more preferably from 2.5% w/w to 12% w/w.

10. The process according to any of the preceding claims, wherein the amount of water is 0.5 to 1.5 mol/mol chlorosulfonyl isocyanate, preferably 0.95 to 1.05 mol/mol chlorosulfonyl isocyanate, most preferably 1 mol/mol chlorosulfonyl isocyanate.

11. The process according to any of the preceding claims, wherein the addition time of water in acetonitrile in the presence of the organic amide or organic amine to chlorosulfonlyl isocyanate in inert solvent is 1 hour to 8 hours, preferably 1 hour to 6 hours, more preferably 1 hour to 4 hours, most preferably 2 hours to 4 hours.

12. The process according to any of the preceding claims, wherein the process is used for converting a compound having structural formula (III) 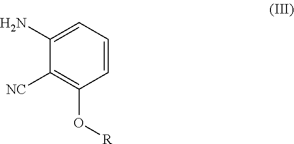
to a compound having structural formula (IV) 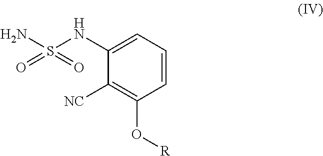
wherein R represents a C₁ to C₂₀ hydrocarbon group optionally comprising one or more heteroatoms such as oxygen, nitrogen and sulfur.

13. The process according to claim 12, wherein the compound having structural formula (III) is 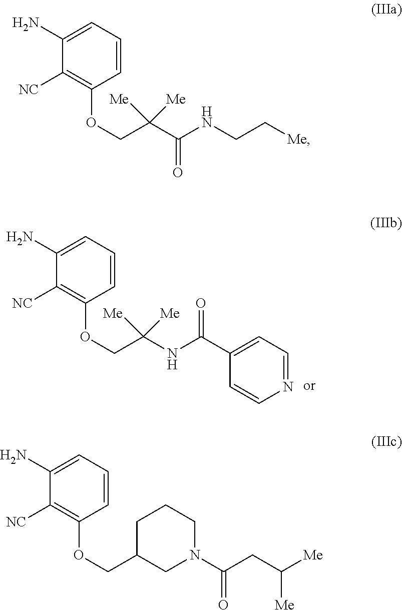 [SMILES: CCCNC(=O)C(C)(C)COc1cccc(N)c1C#N ; CC(C)(COc1cccc(N)c1C#N)NC(=O)c1ccncc1 ; CC(C)CC(=O)N1CCCC(COc2cccc(N)c2C#N)C1]
and wherein the compound having structural formula (IV) is

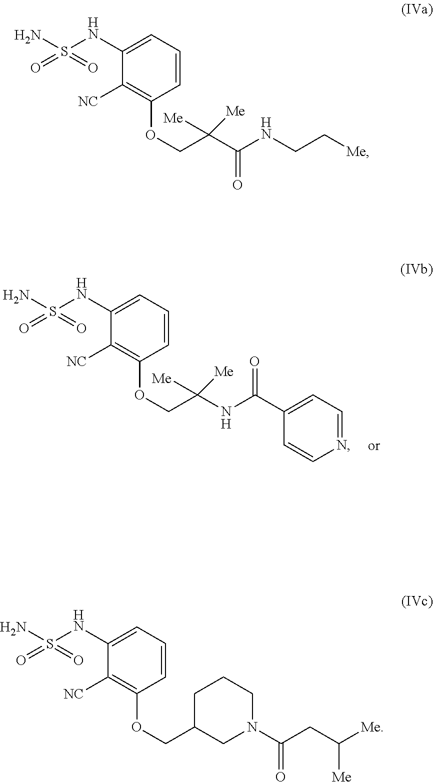 [SMILES: CCCNC(=O)C(C)(C)COc1cccc(NS(N)(=O)=O)c1C#N ; CC(C)(COc1cccc(NS(N)(=O)=O)c1C#N)NC(=O)c1ccncc1 ; CC(C)CC(=O)N1CCCC(COc2cccc(NS(N)(=O)=O)c2C#N)C1]

14. The process according to claims 12 and 13, wherein the compound of structural formula (IV) is converted, preferably in-situ or in a stepwise manner, to a compound of structural formula (V) 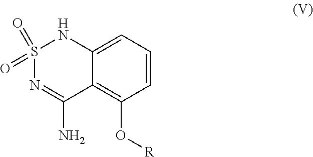
or any orally acceptable salt thereof and
wherein R is defined as in claim 12
or the compound of structural formula (IVa), (IVb) or (IVc) is converted to a compound of structural formula (Va), (Vb) or (Vc) 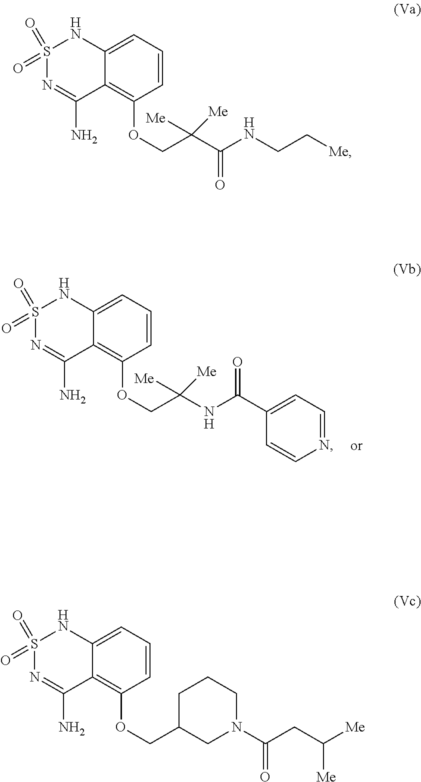 [SMILES: CCCNC(=O)C(C)(C)COc1cccc2c1C(N)=NS(=O)(=O)N2 ; CC(C)(COc1cccc2c1C(N)=NS(=O)(=O)N2)NC(=O)c1ccncc1 ; CC(C)CC(=O)N1CCCC(COc2cccc3c2C(N)=NS(=O)(=O)N3)C1]
or any orally acceptable salt thereof.

15. Use of a compound having structural formula (I) 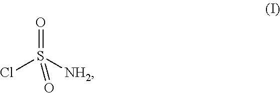
for the preparation of a compound having structural formula (IVa), (IVb) or (IVc) 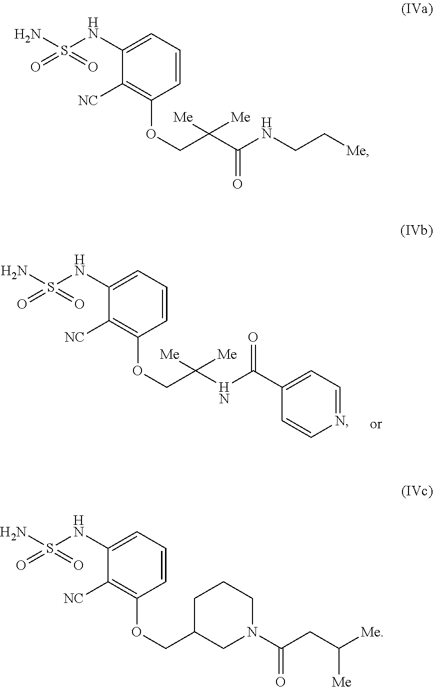 [SMILES: CCCNC(=O)C(C)(C)COc1cccc(NS(N)(=O)=O)c1C#N ; CC(C)(COc1cccc(NS(N)(=O)=O)c1C#N)NC(=O)c1ccncc1 ; CC(C)CC(=O)N1CCCC(COc2cccc(NS(N)(=O)=O)c2C#N)C1]
